(19) United States
(12) Patent Application Publication
(10) Pub. No.: US 2004/0013678 A1
(43) Pub. Date: Jan. 22, 2004

(54) Solid adjuvants

(76) Inventors: Gerhard Schnabel, Elsenfeld (DE); Thomas Maier, Hofheim (DE); Caroline Thuaud, Maisons-Alfort (FR); Hans-Peter Krause, Hofheim (DE); Udo Bickers, Wietmarschen (DE)

Correspondence Address: CONNOLLY BOVE LODGE & HUTZ, LLP, P O BOX 2207, WILMINGTON, DE, 19899, US

(21) Appl. No.: 10/619,065

(22) Filed: Jul. 11, 2003

(30) Foreign Application Priority Data
Jul. 12, 2002 (DE) 102 31 615.5

(51) Int. Cl. A61K 39/00

(52) U.S. Cl. 424/184.1

(57) ABSTRACT

The present invention relates to a solid adjuvant comprising

a) one or more surfactants of the formula (I),

Ar—O—(CHR¹—CHR²—O—)_{y}—R³ (I)

where

Ar is aryl which is substituted by at least two (C₁-C₃₀)alkyl radicals,

R¹ is H or (C₁-C₆)alkyl,

R² is H or (C₁-C₆)alkyl,

R³ is H, an unsubstituted or substituted (C₁-C₃₀) hydrocarbon radical, a sulfonate radical, a phosphonate radical or an acyl radical, and

y is an integer from 1 to 100, and

b) one or more fatty acid esters. The adjuvant is particularly suitable in the field of crop protection.

The present invention relates to novel solid adjuvants, in particular those which are advantageously suitable for the field of crop protection, for example in combination with agrochemical active substances.

The technology of combining agrochemical active substances and adjuvants (materials which enhance their activity, but which are not bioactive themselves)—for example by mixing in the spray tank—is widely used in practice and described intensively in the specialist literature. Most of the cases described deal with liquid adjuvants (see, for example, C. L. Foy, D. W. Pritchard (Ed.), “Pesticide Formulation and Adjuvant Technology”, CRC Press, Inc, 1996, Boca Raton, Fla.,. USA).

It is known that the control of undesired plant growth can be increased by the addition of adjuvants to a multiplicity of agrochemical active substances. Adjuvants in the form of a solid formulation are described in the literature (see, for example, EP 955 810 A1, EP 955 809 A1, EP 968649 A1). However, such adjuvants were economically irrelevant to date since they are expensive, insufficiently storage-stable from the physical point of view or else not sufficiently user friendly, for example insufficient disintegration in the spray mixture or a large volume/weight ratio per unit area.

The object of the present invention was thus to provide novel adjuvants which have advantageous properties, in particular in combination with agrochemical active substances such as herbicides.

Surprisingly, it has now been found that this object is achieved by the specific adjuvants of the present invention.

The present invention thus relates to a solid adjuvant comprising

a) one or more surfactants of the formula (I)

Ar—O—(CHR¹—CHR²—O—)_{y}—R³ (I)

where

Ar is aryl which is substituted by at least two, preferably 2 to 10, (C₁-C₃₀)alkyl radicals,

R¹ is H or (C₁-C₆)alkyl,

R² is H or (C₁-C₆)alkyl,

R³ is H, an unsubstituted or substituted C₁-C₃₀ hydrocarbon radical, preferably (C₁-C₃₀)alkyl, (C₂-C₃₀)alkenyl or (C₂-C₃₀)alkynyl, a sulfonate radical, a phosphonate radical, an acyl radical, and

y is an integer from 1 to 1000, and

b) one or more fillers.

If y>1 in the surfactants of the formula (I), the y units (CHR¹—CHR²—O) can be identical (for example ethylene oxide homopolymer units, propylene oxide homopolymer units or butylene oxide homopolymer units) or different from one another (for example ethylene oxide/propylene oxide copolymer units or ethylene oxide/butylene oxide copolymer units). Surfactants of the formula (I) are generally known, for example from “Presentation about selected Product Groups, Clariant GmbH, Division Surfactants, p.39, September 1997” and also commercially available, for example those from the Sapogenat® T series by Clariant AG. Moreover, surfactants of the formula (I) can be prepared by known reactions, for example surfactants of the formula (I) where R³=H by reaction of commercially available epoxides, for example those of the formula (I′), with hydroxyaromatics, for example those of the formula (I″), under catalytic conditions (for example NaOH and/or sodium acetate; temperature approx. 100-200° C.; superatmospheric pressure of approx. 2-10 bar).

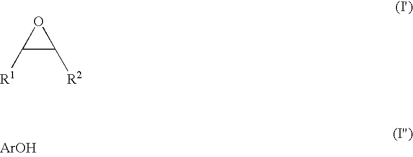

The radicals R¹ and R² in formula (I′) and the radical Ar in formula (I″) are as defined in formula (I). Surfactants of the formula (I) where R³≠H can be obtained from surfactants of the formula (I) where R³=H by standard reactions. For example, surfactants of the formula (I) where R³=(substituted) hydrocarbon radical such as alkyl, alkenyl or alkynyl can be obtained by alkylation, alkenylation or alkynylation, for example with alkyl halides, alkenyl halides or alkynyl halides, with base catalysis; those where R³=sulfonate radical can be obtained by sulfatation followed by neutralization; those where R³=phosphonate radical can be obtained by phosphatation; those where R³=acyl radical can be obtained by acylation.

These reactions are well known to the skilled worker and described, for example, in “Surfactants in Consumer Products” (J. Falbe, Springer Verlag Heidelberg, 1987 and literature cited therein) or J. March, Advanced Organic Chemistry, 4th Edition, John Wiley & Sons, New York, 1992.

The epoxides of the formula (I′) can be obtained by known methods, for example from the corresponding alkenes, and are commercially available, for example ethylene oxide or propylene oxide.

The compounds of the formula (I″) are commercially available and described in the literature; likewise, they can be prepared by standard reactions with which the skilled worker is familiar. Thus, for example, hydroxyaromatics, e.g. phenol, can be reacted with alcohols, olefins or alkyl halides under catalytic conditions (protic acids such as sulfuric or phosphoric acid, or Lewis acids such as aluminum chloride or boron trifluoride diethyl ether) to give the compounds of the formula (I″). A broad overview can be found in, for example, “Methoden der organischen Chemie” [Methods in organic chemistry] (Houben-Weyl), 4th Edition, 1976, Vol. 6/1c, p. 925 et seq.; (ISBN 3-13-204204-8).

Preferred surfactants are those of the formula (I) where Ar in formula (I) is a naphthyl or phenyl radical which has 3 to 7, (C₁-C₁₀)alkyl radicals attached to it. Ar is preferably tri(C₁-C₆)alkylphenyl, especially preferably tributylphenyl such as tri-2,4,6-sec-butylphenyl. R¹ and R² are preferably H or methyl, especially preferably H.

R³ is preferably H, (C₁-C₂₂)alkyl, (C₂-C₂₂)alkenyl, (C₂-C₂₂)alkynyl, an acyl radical such as CO—(C₁-C₃₀)alkyl, CO—(C₂-C₃₀)alkenyl, CO—(C₂-C₃₀)alkynyl, CO—(C₁-C₃₀)alkoxy, CO—(C₂-C₃₀)alkenyloxy, CO—(C₂-C₃₀)alkynyloxy or COH, or a sulfonate radical such as SO₃M where M is a cation, such as an inorganic cation, for example an alkali metal or alkaline earth metal cation such as Na, K or Mg, or an organic cation, for example a primary, secondary, tertiary or quaternary ammonium ion such as NH₃CH₃, NH₂(CH₃)₂, NH(C₂H₅)₃ or N(CH₃)₄, or a phosphonate radical such as (O)P(OR′) (OR″), where R′, R″ independently of one another are H or a cation such as an inorganic cation, for example an alkali metal or alkaline earth metal cation such as Na, K or Mg or an organic cation, for example a primary, secondary, tertiary or quaternary ammonium ion such as NH₃CH₃, NH₂(CH₅)₂, NH(C₂H₃)₃ or N(CH₃)₄, it also being possible for R′, R″ to be Ar—O—(CHR¹CHR²)_{y} where Ar, R¹, R² and y are as defined in formula (I). R³ is especially preferably H, (C₁-C₆)alkyl or SO₃M, where M is a cation.

Values of from 15 to 100 are preferred for y, with values of from 30 to 80 being especially preferred.

Very especially preferred surfactants of the formula (I) are those where Ar is tri(C₁-C₆)alkylphenyl, especially preferably tributylphenyl such as tri-2,4,6-sec-butylphenyl, R¹=R²=R³=H and y is an integer of from 30 to 80, for example surfactants from the Sapogenat® T series by Clariant, for example Sapogenat® T 180, Sapogenat® T 300 and Sapogenat® T 500. Preferred surfactants of the formula (I) are solid under standard conditions (room temperature, atmospheric pressure).

In formula (I) and all other formulae of the present description, carbon-containing radicals such as alkyl, alkoxy, haloalkyl, haloalkoxy, alkylamino and alkylthio and the corresponding unsaturated and/or substituted radicals can be in each case straight-chain or branched in the carbon skeleton. Unless specifically stated, these radicals generally have 1 to 30 carbon atoms, the lower carbon skeletons, for example those having 1 to 6 carbon atoms or, in the case of unsaturated groups, those having 2 to 6 carbon atoms, being preferred. Alkyl radicals, also in the composite meanings such as alkoxy, haloalkyl and the like, are, for example, methyl, ethyl, n- or i-propyl, n-, i-, t- or sec-butyl, pentyl radicals, hexyl radicals such as n-hexyl, i-hexyl and 1,3-dimethylbutyl, heptyl radicals, such as n-heptyl, 1-methylhexyl and 1,4-dimethylpentyl; alkenyl and alkynyl radicals have the meanings of the possible unsaturated radicals which correspond to the alkyl radicals; alkenyl is, for example, allyl, 1-methylprop-2-en-1-yl, 2-methylprop-2-en-1-yl, but-2-en-1-yl, but-3-en-1-yl, 1-methylbut-3-en-1-yl and 1-methylbut-2-en-1-yl; alkynyl is, for example, propargyl, but-2-yn-1-yl, but-3-yn-1-yl, 1-methylbut-3-yn-1-yl.

Alkenyl in the form (C₃-C₄)alkenyl, (C₃-C₅)alkenyl, (C₃-C₆)alkenyl, (C₃-C₈)alkenyl or (C₃-C₁₂)alkenyl is preferably an alkenyl radical having 3 to 4, 3 to 5, 3 to 6, 3 to 8 and 3 to 12 carbon atoms, respectively, where the double bond is not located at the carbon atom which is linked to the remaining moiety of the compound of the formula (I) (“yl” position). This also applies analogously to (C₃-C₄)alkynyl and the like, (C₃-C₄)alkenyloxy and the like and (C₃-C₄)alkynyloxy and the like.

A hydrocarbon radical means a straight-chain, branched or cyclic and saturated or unsaturated aliphatic or aromatic hydrocarbon radical, for example alkyl, alkenyl, alkynyl, cycloalkyl, cycloalkenyl or aryl. A hydrocarbon radical has preferably 1 to 40 carbon atoms, preferably 1 to 30 carbon atoms; a hydrocarbon radical is especially preferably alkyl, alkenyl or alkynyl having up to 12 carbon atoms or cycloalkyl having 3, 4, 5, 6 or 7 ring atoms, or phenyl.

Aryl is a mono-, bi- or polycyclic aromatic system, for example phenyl, naphthyl, tetrahydronaphthyl, indenyl, indanyl, pentalenyl, fluorenyl and the like, preferably phenyl.

A heterocyclic radical or ring (heterocyclyl) can be saturated, unsaturated or heteroaromatic and unsubstituted or substituted; preferably, it contains one or more hetero atoms in the ring, preferably selected from the group consisting of N, O and S; it is preferably an aliphatic heterocyclyl radical having 3 to 7 ring atoms or a heteroaromatic radical having 5 or 6 ring atoms and contains 1, 2 or 3 hetero atoms. The heterocyclic radical can be, for example, a heteroaromatic radical or ring (heteroaryl) such as, for example, a mono-, bi- or polycyclic aromatic system in which at least 1 ring contains one or more hetero atoms, for example pyridyl, pyrimidinyl, pyridazinyl, pyrazinyl, triazinyl, thienyl, thiazolyl, oxazolyl, furyl, pyrrolyl, pyrazolyl and imidazolyl, or a partially or fully hydrogenated radical such as oxiranyl, oxetanyl, pyrrolidyl, piperidyl, piperazinyl, dioxolanyl, morpholinyl, tetrahydrofuryl. Suitable substituents for a substituted heterocyclic radical are those substituents which are mentioned hereinbelow, and additionally oxo. The oxo group may also occur on those hetero ring atoms which may exist in different oxidation states, for example N and S.

Halogen is, preferably, fluorine, chlorine, bromine or iodine. Haloalkyl, -alkenyl and -alkynyl are alkyl, alkenyl or alkynyl which are substituted in part or fully by halogen, preferably by fluorine, chlorine and/or bromine, in particular by fluorine or chlorine, e.g. CF₃, CHF₂, CH₂F, CF₃CF₂, CH₂FCHCl, CCl₃, CHCl₂, CH₂CH₂Cl; haloalkoxy is, for example, OCF₃, OCHF₂, OCH₂F, CF₃CF₂O, OCH₂CF₃ and OCH₂CH₂Cl; this also applies analogously to haloalkenyl and other halogen-substituted radicals.

Substituted radicals such as substituted hydrocarbon radicals, e.g. substituted alkyl, alkenyl, alkynyl, aryl, phenyl and benzyl, or substituted heterocyclyl or heteroaryl, are, for example, a substituted radical which is derived from the unsubstituted skeleton, where the substituents are, for example, one or more, preferably 1, 2 or 3, radicals selected from the group consisting of halogen, alkoxy, haloalkoxy, alkylthio, hydroxyl, amino, nitro, carboxyl, cyano, azido, alkoxycarbonyl, alkylcarbonyl, formyl, carbamoyl, mono- and dialkylaminocarbonyl, substituted amino such as acylamino, mono- and dialkylamino, and alkylsulfinyl, haloalkylsulfinyl, alkylsulfonyl, haloalkylsulfonyl and, in the case of cyclic radicals, also alkyl and haloalkyl, and unsaturated aliphatic radicals which correspond to the abovementioned saturated hydrocarbon-containing radicals, such as alkenyl, alkynyl, alkenyloxy, alkynyloxy and the like. In the case of radicals with carbon atoms, those having 1 to 4 carbon atoms, in particular 1 or 2 carbon atoms, are preferred. Preferred substituents are, as a rule, those from the groups consisting of halogen, for example fluorine and chlorine, (C₁-C₄)alkyl, preferably methyl or ethyl, (C₁-C₄)haloalkyl, preferably trifluoromethyl, (C₁-C₄)alkoxy, preferably methoxy or ethoxy, (C₁-C₄)haloalkoxy, nitro and cyano. Especially preferred in this context are the substituents methyl, methoxy and chlorine.

Optionally substituted phenyl is preferably phenyl which is unsubstituted or mono- or polysubstituted, preferably up to trisubstituted, by identical or different radicals selected from the group consisting of halogen, (C₁-C₄)alkyl, (C₁-C₄)alkoxy, (C₁-C₄)haloalkyl, (C₁-C₄)haloalkoxy and nitro, for example o-, m- and p-tolyl, dimethylphenyl radicals, 2-, 3- and 4-chlorophenyl, 2-, 3- and 4-trifluoro- and -trichlorophenyl, 2,4-, 3,5-, 2,5- and 2,3-dichlorophenyl, o-, m- and p-methoxyphenyl, or 2,4,6-tributylphenyl such as 2,4,6-tri-sec-butylphenyl.

An acyl radical refers to the radical of an organic acid which is formed formally by removing an OH group from the organic acid, for example the radical of a carboxylic acid and radicals of acids derived therefrom, such as thiocarboxylic acid, unsubstituted or N-substituted iminocarboxylic acids or the radicals of carbonic monoesters, unsubstituted or N-substituted carbamic acids, sulfonic acids, sulfinic acids, phosphonic acids, phosphinic acids.

An acyl radical is preferably formyl or aliphatic acyl selected from the group consisting of CO—R^{X}, CS—R^{X}, CO—OR^{X}, CS—OR^{X}, CS—SR^{X}, SOR^{Y} or SO₂R^{Y}, where R^{X} and R^{Y} each are a C₁-C₃₀ hydrocarbon radical which is unsubstituted or substituted, or aminocarbonyl or aminosulfonyl, the last-mentioned two radicals being unsubstituted, N-monosubstituted or N,N-disubstituted. Acyl is, for example, formyl, haloalkylcarbonyl, alkylcarbonyl such as (C₁-C₄)alkylcarbonyl, phenylcarbonyl, it being possible for the phenyl ring to be substituted, for example as stated above for phenyl, or is alkyloxycarbonyl, phenyloxycarbonyl, benzyloxycarbonyl, alkylsulfonyl, alkylsulfinyl, N-alkyl-1-iminoalkyl and other radicals of organic acids.

Formula (I) and the other formulae in the present description also encompass all the stereoisomers and their mixtures. Such compounds contain one or more asymmetric carbon atoms or else double bonds which are not stated separately in the general formula. The possible stereoisomers, which are defined by their specific spatial form, such as enantiomers, diastereomers, Z and E isomers, are all embraced by the formulae in question and can be obtained by customary methods from mixtures of the stereoisomers or else by stereoselective reactions in combination with the use of stereochemically pure starting materials.

The fillers b) which are present in the adjuvants according to the present invention are solids. They are generally known, for example, from: W. van Falkenburg (ed.), Pesticide Formulations, Marcel Dekker, Inc., New York, 1973; or from: Schriftenreihe Degussa No. 1, Synthetische Kieselsäuren für Pflanzenschutz- und Schädlingsbekämpfungsmittel [Synthetic silicas for crop protection products and pesticides], March 1989. They are also commercially available.

Preferred as fillers b) are, for example, inorganic and organic carriers such as polymeric carbohydrates or silicates. Examples of polymeric carbohydrates are cellulose and its derivatives such as Tylose®, Tylopur®, Methylan® and Finnix®, or starch and its derivatives such as Maizena® and Mondamin®. Silicates can be of natural or synthetic origin. Examples of natural silicates are kaolin, bentonite, talc, pyrophyllite or diatomaceous earth. Examples of synthetic silicates are pyrogenic silicas or precipitated silicas, for example Sipernat® (for example Sipernat® 50 S or Sipernat® 500 LS), Dessalon®, Aerosil®, Silkasil® or Ketiensil®. For the purposes of the present invention, the term silicates also encompasses silicates such as earth metal silicates and alkaline earth metal silicates, for example alumosilicates or magnesium silicates.

The adjuvants according to the present invention are solid under standard conditions (atmospheric pressure, room temperature). Adjuvants according to the present invention may be present for example in the form of granules, powders or dusts. In general, they contain 10-80% by weight, preferably 25-75% by weight, especially preferably 40-70% by weight, of one or more surfactants of the formula (I) and 90-20% by weight, preferably 75-25% by weight, especially preferably 60-30% by weight, of fillers b). In individual cases, these limits may also be lower or higher. Preferred adjuvants according to the present invention comprise the surfactants of the formula (I) in an excess relative to the fillers b).

In addition, the adjuvants according to the invention may comprise typical auxiliaries such as additives and formulation aids. The term auxiliaries is understood as meaning materials which are largely inert chemically and biologically, and whose use allows a composition to be handled as intended.

Examples of auxiliaries are

wetting agents such as Genapol® LRO (0-20% by weight), dispersants such as Tamol® (0-15% by weight) or other surfactants (nonionic, cationic, anionic, polymeric surfactants) (0-30% by weight);

inorganic salts such as NaCl, Na₂SO₄, MgCl₂ (0-50% by weight), (oligo-, poly-)phosphates; carbonates such as potassium carbonate;

fertilizers such as ammonium sulfate, ammonium nitrate, urea, phosphorus- and potassium-containing components, if desired other trace elements (0-60% by weight);

antifoams such as Fluowet® PP (0-2% by weight);

disintegrants such as, for example, effervescent powder (citric acid/NaHCO₃) (0-20% by weight), microcrystalline cellulose, polyvinylpyrrolidone;

binders such as suitable natural or synthetic materials, such as polyamino acids, polyvinyl alcohols, polyvinylpyrrolidone, polyacrylic acid derivatives (0-15% by weight); or

solvents such as water or organic solvents (0-15% by weight).

The amounts (% by weight) given for the auxiliaries are ranges which are regarded as typical, but can also be higher or lower in individual cases.

The adjuvants according to the present invention can be prepared by known methods (cf. Hans Mollet, Arnold Grubenmann, Formuliertechnik [Formulation technology]; Wiley-VCH, Weinheim, 2000; p.183 et seq. and literature cited therein). The adjuvants may be present for example in the form of granules, powders or dusts. Granules can be obtained for example by melt extrusion followed by granulation. Powders or dusts can be obtained by reducing precomminuted extrudates to the desired particle size, for example using an air-jet mill.

The adjuvants according to the invention can be combined with one or more agrochemical active substances to give agrochemical compositions, for example in the form of coformulations or tank mixes. Such agrochemical compositions are likewise novel and subject of the present invention.

Examples of suitable agrochemical active substances are herbicides, insecticides, fungicides, safeners and growth regulators. Preferred agrochemical active substances are herbicides, for example foliar-acting herbicides such as ALS inhibitors (for example sulfonamides such as flucarbazone, propoxycarbazone or amicarbazone, or sulfonylureas such as mesosulfuron, ethoxysulfuron, iodosulfuron, amidosulfuron, foramsulfuron), diflufenican, bromoxynil- or ioxynil-containing products, herbicides from the class of the aryloxyphenoxypropionates such as fenoxaprop-p-ethyl, sugarbeet herbicides such as desmedipham, phenmedipham, ethofumesate or metamitron, glyphosate or glufosinate or else active substances from the class of the HPPD inhibitors (for example isoxaflutole, sulcotrione, mesotrione)

Herbicides which are present in the agrochemical compositions according to the invention are, for example, ALS inhibitors (acetolactate synthetase inhibitors), or herbicides other than ALS inhibitors, such as herbicides from the group of the carbamates, thiocarbamates, haloacetanilides, substituted phenoxy-, naphthoxy- and phenoxyphenoxycarboxylic acid derivatives and heteroaryloxyphenoxyalkanecarboxylic acid derivatives such as quinolyloxy-, quinoxalyloxy-, pyridyloxy-, benzoxazolyloxy- and benzothiazolyloxyphenoxyalkanecarboxylic esters, cyclohexanedione derivatives, phosphorus-containing herbicides, for example herbicides of the glufosinate type or of the glyphosate type, and S-(N-aryl-N-alkylcarbamoylmethyl)dithiophosphoric acid esters.

The ALS inhibitors are, in particular, imidazolinones, pyrimidinyloxypyridinecarboxylic acid derivatives, pyrimidyloxybenzoic acid derivatives, triazolopyrimidinesulfonamide derivatives and sulfonamides, preferably from the group of the sulfonylureas, particularly preferably those of the general formula (II) and/or their salts,

R^{α}—SO₂—NR^{β}—CO—(NR^{γ})ₓ—R^{δ} (II)

where

R^{α} is a hydrocarbon radical, preferably an aryl radical such as phenyl, which is unsubstituted or substituted, or a heterocyclic radical, preferably a heteroaryl radical such as pyridyl, which is unsubstituted or substituted, and where the radicals including substituents have 1-30 carbon atoms, preferably 1-20 carbon atoms, or R^{α} is an electron-attracting group such as a sulfonamide radical,

R^{β} is a hydrogen atom or a hydrocarbon radical which is unsubstituted or substituted and, including substituents, has 1-10 carbon atoms, for example unsubstituted or substituted C₁-C₆alkyl, preferably a hydrogen atom or methyl,

R^{γ} is a hydrogen atom or a hydrocarbon radical which is unsubstituted or substituted and, including substituents, has 1-10 carbon atoms, for example unsubstituted or substituted C₁-C₆alkyl, preferably a hydrogen atom or methyl,

x is zero or 1, and

R^{δ} is a heterocyclic radical.

Especially preferred ALS inhibitors are sulfonylureas of the formula (III) and/or their salts,

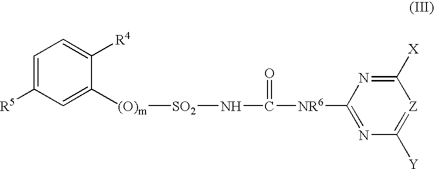

where

R⁴ is C₁-C₄alkoxy, preferably C₂-C₄alkoxy, or CO—R^{a} where R^{a} is OH, C₁-C₄alkoxy or NR^{b}R^{c}, where R^{b} and R^{c} independently of one another are identical or different and are H or C₁-C₄alkyl,

R⁵ is halogen or (A)ₙ—NR^{d}R^{e} where n is zero or 1, A is a group CR′R″ where R′ and R″ independently of one another are identical or different and are H or C₁-C₄alkyl, R^{d} is H or C₁-C₄alkyl and R^{e} is an acyl radical such as formyl or C₁-C₄alkylsulfonyl, and, in the event that R⁴ is C₁-C₄alkoxy, preferably C₂-C₄alkoxy, R⁵ may also be H,

R⁶ is H or C₁-C₄alkyl,

m is zero or 1, preferably zero,

X and Y independently of one another are identical or different and are C₁-C₆alkyl, C₁-C₆alkoxy or C₁-C₆alkylthio, where each of the three radicals mentioned is unsubstituted or substituted by one or more radicals selected from the group consisting of halogen, C₁-C₄alkoxy and C₁-C₄alkylthio, or are C₃-C₆cycloalkyl, C₂-C₆alkenyl, C₂-C₆alkynyl, C₃-C₆alkenyloxy or C₃-C₆alkynyloxy, preferably C₁-C₄alkyl or C₁-C₄alkoxy, and

Z is CH or N.

Preferred sulfonylureas of the formula (III) and/or their salts are those in which

m is zero and

a) R⁴ is CO—(C₁-C₄alkoxy) and R⁵ is halogen, preferably iodine, or R⁵ is CH₂—NHR^{e} where R^{e} is an acyl radical, preferably C₁-C₄-alkylsulfonyl, or

b) R⁴ is CO—N(C₁-C₄alkyl)₂ and R⁵ is NHR^{e} where R^{e} is an acyl radical, preferably formyl.

For the purposes of the present invention, the active substances from the group of the ALS inhibitors, such as sulfonylureas, which are present as component in the herbicidal compositions according to the invention are always also understood as meaning not only the neutral compounds, but also their salts with inorganic and/or organic counterions.

Thus, for example, sulfonylureas may form salts in which the hydrogen of the —SO₂—NH— group is replaced by an agriculturally suitable cation. Examples of these salts are metal salts, in particular alkali metal salts or alkaline earth metal salts, in particular sodium and potassium salts, or else ammonium salts or salts with organic amines. Likewise, salt formation may take place by addition of an acid to basic groups such as, for example, amino and alkylamino. Acids which are suitable for this purpose are strong inorganic and organic acids, for example HCl, HBr, H₂SO₄ or HNO₃.

Preferred ALS inhibitors are from the series of the sulfonylureas, for example pyrimidine- or triazinylaminocarbonyl[benzene-, pyridine-, pyrazole-, thiophene- and (alkylsulfonyl)alkylamino]sulfamides. Preferred as substituents on the pyrimidine ring or triazine ring are alkoxy, alkyl, haloalkoxy, haloalkyl, halogen or dimethylamino, it being possible for all substituents to be combined independently of one another. Preferred substituents in the benzene-, pyridine-, pyrazole-, thiophene- or (alkylsulfonyl)alkylamino moiety are alkyl, alkoxy, halogen such as F, Cl, Br or I, amino, alkylamino, dialkylamino, acylamino such as formylamino, nitro, alkoxycarbonyl, aminocarbonyl, alkylaminocarbonyl, dialkylaminocarbonyl, alkoxyaminocarbonyl, haloalkoxy, haloalkyl, alkylcarbonyl, alkoxyalkyl, alkylsulfonylaminoalkyl, (alkanesulfonyl)alkylamino. Examples of such suitable sulfonylureas are

A1) Phenyl- and benzylsulfonylureas and related compounds, for example

1-(2-chlorophenylsulfonyl)-3-(4-methoxy-6-methyl-1,3,5-triazin-2-yl )urea (chlorsulfuron),

1-(2-ethoxycarbonylphenylsulfonyl)-3-(4-chloro-6-methoxypyrimidin-2-yl )urea (chlorimuron-ethyl),

1-(2-methoxyphenylsulfonyl)-3-(4-methoxy-6-methyl-1,3,5-triazin-2-yl)urea (metsulfuron-methyl),

1-(2-chloroethoxyphenylsulfonyl)-3-(4-methoxy-6-methyl-1,3,5-triazin-2-yl)urea (triasulfuron),

1-(2-methoxycarbonylphenylsulfonyl)-3-(4,6-dimethylpyrimidin-2-yl)urea (sulfumeturon-methyl),

1-(2-methoxycarbonylphenylsulfonyl)-3-(4-methoxy-6-methyl-1,3,5-triazin-2-yl )-3-methylurea (tribenuron-methyl),

1-(2-methoxycarbonylbenzylsulfonyl)-3-(4,6-dimethoxypyrimidin-2-yl )urea (bensulfuron-methyl),

1-(2-methoxycarbonylphenylsulfonyl)-3-(4,6-bis(difluoromethoxy)pyrimidin-2-yl)urea (primisulfuron-methyl),

3-(4-ethyl-6-methoxy-1,3,5-triazin-2-yl)-1-(2,3-dihydro-1,1-dioxo-2-methylbenzo[b]thiophen-7-sulfonyl)urea (EP-A 0 796 83),

3-(4-ethoxy-6-ethyl-1,3,5-triazin-2-yl)-1-(2,3-dihydro-1,1-dioxo-2-methylbenzo[b]thiophen-7-sulfonyl)urea (EP-A 0 079 683),

3-(4-methoxy-6-methyl-1,3,5-triazin-2-yl)-1-(2-methoxycarbonyl-5-iodophenylsulfonyl)urea (iodosulfuron-methyl and its salts such as the sodium salt, WO 92/13845),

DPX-66037, triflusulfuron-methyl (see Brighton Crop Prot. Conf.—Weeds—1995, p. 853),

CGA-277476, (see Brighton Crop Prot. Conf.—Weeds—1995, p. 79),

methyl 2-[3-(4,6-dimethoxypyrimidin-2-yl)ureidosulfonyl]-4-methanesulfonamido-methylbenzoate (mesosulfuron-methyl and its salts such as the sodium salt, WO 95/10507),

N,N-dimethyl-2-[3-(4,6-dimethoxypyrimidin-2-yl)ureidosulfonyl]-4-formylamino-benzamide (foramsulfuron and its salts such as the sodium salt, WO 95/01344);

A2) Thienylsulfonylureas, for example

1-(2-methoxycarbonylthiophen-3-yl)-3-(4-methoxy-6-methyl-1,3,5-triazin-2-yl)urea (thifensulfuron-methyl);

A3) Pyrazolylsulfonylureas, for example

1-(4-ethoxycarbonyl-1-methylpyrazol-5-ylsulfonyl)-3-(4,6-dimethoxypyrimidin-2-yl)urea (pyrazosulfuron-methyl);

methyl 3-chloro-5-(4,6-dimethoxypyrimidin-2-ylcarbamoylsulfamoyl)-1-methylpyrazole-4-carboxylate (EP-A 0 282 613);

methyl 5-(4,6-dimethylpyrimidin-2-ylcarbamoylsulfamoyl)-1-(2-pyridyl)pyrazole-4-carboxylate (NC-330, see Brighton Crop Prot. Conference ‘Weeds’ 1991, Vol. 1, p. 45 et seq.),

DPX-A8947, azimsulfuron, (see Brighton Crop Prot. Conf. ‘Weeds’ 1995, p. 65);

A4) Sulfone diamide derivatives, for example

3-(4,6-dimethoxypyrimidin-2-yl)-1-(N-methyl-N-methylsulfonylaminosulfonyl)urea (amidosulfuron) and its structural analogs (EP-A 0 131 258 and Z. Pfl. Krankh. Pfl. Schutz, Special Issue XII, 489-497 (1990));

A5) Pyridylsulfonylureas, for example

1-(3-N,N-dimethylaminocarbonylpyridin-2-ylsulfonyl)-3-(4,6-dimethoxypyrimidin-2-yl)urea (nicosulfuron),

1-(3-ethylsulfonylpyridin-2-ylsulfonyl)-3-(-(4,6-dimethoxypyrimidin-2-yl )urea (rimsulfuron),

methyl 2-[3-(4,6-dimethoxypyrimidin-2-yl)ureidosulfonyl]-6-trifluoromethyl-3-pyridinecarboxylate, sodium salt (DPX-KE 459, flupyrsulfuron, see Brighton Crop Prot. Conf. Weeds, 1995, p. 49),

pyridylsulfonylureas as are described, for example in DE-A 40 00 503 and DE-A 40 30 577, preferably those of the formula

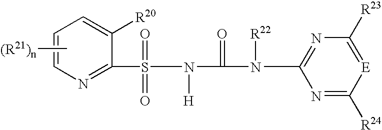

in which

E is CH or N, preferably CH,

R²⁰ is iodine or NR²⁵R²⁶,

R²¹ is hydrogen, halogen, cyano, (C₁-C₃)alkyl, (C₁-C₃)alkoxy, (C₁-C₃)haloalkyl, (C₁-C₃)haloalkoxy, (C₁-C₃)alkylthio, (C₁-C₃)alkoxy(C₁-C₃)alkyl, (C₁-C₃)alkoxycarbonyl, mono- or di((C₁-C₃)alkyl)amino, (C₁-C₃)alkylsulfinyl or -sulfonyl, SO₂—NR^{x}R^{y} or CO—NR^{x}R^{y}, in particular hydrogen,

R^{x}, R^{y} independently of one another are hydrogen, (C₁-C₃)alkyl, (C₁-C₃)alkenyl, (C₁-C₃)alkynyl or together are —(CH₂)₄—, —(CH₂)₅— or —(CH₂)₂—O—(CH₂)₂—,

n is 0, 1, 2 or 3, preferably 0 or 1,

R²² is hydrogen or CH₃,

R²³ is halogen, (C₁-C₂)alkyl, (C₁-C₂)alkoxy, (C₁-C₂)haloalkyl, in particular CF₃, (C₁-C₂)haloalkoxy, preferably OCHF₂ or OCH₂CF₃,

R²⁴ is (C₁-C₂)alkyl, (C₁-C₂)haloalkoxy, preferably OCHF₂, or (C₁-C₂)alkoxy,

R²⁵ is (C₁-C₄)alkyl,

R²⁶ is (C₁-C₄)alkylsulfonyl or

R²⁵ and R²⁶ together are a chain of the formula —(CH₂)₃SO₂— or —(CH₂)₄SO₂—, for example 3-(4,6-dimethoxypyrimiden-2-yl)-1-(3-N-methylsulfonyl-N-methylaminopyridin-2-yl)sulfonylurea, or their salts;

A6) Alkoxyphenoxysulfonylureas as are described, for example, in EP-A 0 342 569, preferably those of the formula

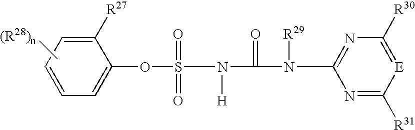

in which

E is CH or N, preferably CH,

R²⁷ is ethoxy, propoxy or isopropoxy,

R²⁸ halogen, NO₂, CF₃, CN, (C₁-C₄)alkyl, (C₁-C₄)alkoxy, (C₁-C₄)alkylthio or (C₁-C₃)alkoxycarbonyl, preferably in the 6-position on the phenyl ring,

n is 0, 1, 2 or 3, preferably 0 or 1,

R²⁹ is hydrogen, (C₁-C₄)alkyl or (C₃-C₄)alkenyl,

R³⁰, R³¹ independently of one another are halogen, (C₁-C₂)alkyl, (C₁-C₂)alkoxy, (C₁-C₂)haloalkyl, (C₁-C₂)haloalkoxy or (C₁-C₂)alkoxy(C₁-C₂)alkyl, preferably OCH₃ or CH₃, for example 3-(4,6-dimethoxypyrimidin-2-yl)-1-(2-ethoxyphenoxy)sulfonylurea, or their salts;

A7) Imidazolylsulfonylureas, for example

MON 37500, sulfosulfuron (see Brighton Crop Prot. Conf. ‘Weeds’, 1995, p. 57), and other related sulfonylurea derivatives and mixtures of these.

Typical representatives of these active substances are, inter alia, the compounds listed hereinbelow: amidosulfuron, azimsulfuron, bensulfuron-methyl, chlorimuron-ethyl, chlorsulfuron, cinosulfuron, cyclosulfamuron, ethametsulfuron-methyl, ethoxysulfuron, flazasulfuron, flupyrsulfuron-methyl-sodium, halosulfuron-methyl, imazosulfuron, metsulfuron-methyl, nicosulfuron, oxasulfuron, primisulfuron-methyl, prosulfuron, pyrazosulfuron-ethyl, rimsulfuron, sulfometuron-methyl, sulfosulfuron, thifensulfuron-methyl, triasulfuron, tribenuron-methyl, triflusulfuron-methyl, iodosulfuron-methyl and its sodium salt (WO 92/13845), mesosulfuron-methyl and its sodium salt (Agrow No. 347, Mar. 3, 2000, page 22 (PJB Publications Ltd. 2000)) and foramsulfuron and its sodium salt (Agrow No. 338, Oct. 15, 1999, page 26 (PJB Publications Ltd. 1999)).

The active substances listed hereinabove are known, for example, from “The Pesticide Manual”, 12^{th} Edition (2000), The British Crop Protection Council, or the references cited after the individual active substances.

Other suitable ALS inhibitors are, for example

B) Imidazolinones, for example

methyl 2-(4-isopropyl-4-methyl-5-oxo-2-imidazolin-2-yl)-5-methylbenzoate and 2-(4-isopropyl-4-methyl-5-oxo-2-imidazolin-2-yl)-4-methylbenzoic acid (imazamethabenz),

5-ethyl-2-(4-isopropyl-4-methyl-5-oxo-2-imidazolin-2-yl)pyridine-3-carboxylic acid (imazethapyr),

2-(4-isopropyl-4-methyl-5-oxo-2-imidazolin-2-yl)quinoline-3-carboxylic acid (imazaquin),

2-(4-isopropyl-4-methyl-5-oxo-2-imidazolin-2-yl)pyridine-3-carboxylic acid (imazapyr),

5-methyl-2-(4-isopropyl-4-methyl-5-oxo-2-imidazolin-2-yl)pyridine-3-carboxylic acid (imazethamethapyr);

C) Triazolopyrimidinesulfonamide derivatives, for example

N-(2,6-difluorophenyl)-7-methyl-1,2,4-triazolo[1,5-c]pyrimidine-2-sulfonamide (flumetsulam),

N-(2,6-dichloro-3-methylphenyl)-5,7-dimethoxy-1,2,4-triazolo[1,5-c]pyrimidine-2-sulfonamide,

N-(2,6-difluorophenyl)-7-fluoro-5-methoxy-1,2,4-triazolo[1,5-c]pyrimidine-2-sulfonamide,

N-(2,6-dichloro-3-methylphenyl)-7-chloro-5-methoxy-1,2,4-triazolo[1,5-c]pyrimidine-2-sulfonamide,

N-(2-chloro-6-methoxycarbonyl)-5,7-dimethyl-1,2,4-triazolo[1,5-c]pyrimidine-2-sulfonamide (EP-A 0 343 752, U.S. Pat. No. 4,988,812);

D) Pyrimidinyloxypyridinecarboxylic acid and pyrimidinyloxybenzoic acid derivatives, for example

benzyl 3-(4,6-dimethoxypyrimidin-2-yl)oxypyridine-2-carboxylate (EP-A 0 249 707),

methyl 3-(4,6-dimethoxypyrimidin-2-yl)oxypyridine-2-carboxylate (EP-A 0 249 707),

2,6-bis[(4,6-dimethoxypyrimidin-2-yl)oxy]benzoic acid (EP-A 0 321 846),

1-(ethoxycarbonyloxyethyl)2,6-bis[(4,6-dimethoxypyrimidin-2-yl)oxy]benzoate (EP-A 0 472 113).

The herbicidal active substances which are present in the herbicidal compositions according to the invention and which differ from the ALS inhibitors are, for example, herbicides from the group of the carbamates, thiocarbamates, haloacetanilides, substituted phenoxy-, naphthoxy- and phenoxyphenoxycarboxylic acid derivatives, and heteroaryloxyphenoxyalkanecarboxylic acid derivatives such as quinolyloxy-, quinoxalyloxy-, pyridyloxy-, benzoxazolyloxy- and benzothiazolyloxyphenoxyalkanecarboxylic esters, cyclohexanedione derivatives, phosphorus-containing herbicides, for example of the glufosinate type or of the glyphosate type, and S-(N-aryl-N-alkylcarbamoylmethyl)dithiophosphoric esters. Preferred in this context are phenoxyphenoxy- and heteroaryloxyphenoxycarboxylic esters and their salts, and herbicides such as bentazone, cyanazine, atrazine, dicamba or hydroxybenzonitriles such as bromoxynil and ioxynil and other foliar-acting herbicides.

Suitable herbicidal active substances which differ from the ALS inhibitors and which may be present as a component in the agrochemical compositions according to the invention are, for example:

E) Herbicides of the phenoxyphenoxy- and heteroaryloxyphenoxycarboxylic acid derivatives type, such as

E1) Phenoxyphenoxy- and benzyloxyphenoxycarboxylic acid derivatives, for example methyl 2-(4-(2,4-dichlorophenoxy)phenoxy)propionate (diclofop-methyl),

methyl 2-(4-(4-bromo-2-chlorophenoxy)phenoxy)propionate (DE-A 26 01 548),

methyl 2-(4-(4-bromo-2-fluorophenoxy)phenoxy)propionate (U.S. Pat. No. 4,808,750),

methyl 2-(4-(2-chloro-4-trifluoromethylphenoxy)phenoxy)propionate (DE-A 24 33 067),

methyl 2-(4-(2-fluoro-4-trifluoromethylphenoxy)phenoxy)propionate (U.S. Pat. No. 4,808,750),

methyl 2-(4-(2,4-dichlorobenzyl)phenoxy)propionate (DE-A 24 17 487),

ethyl 4-(4-(4-trifluoromethylphenoxy)phenoxy)pent-2-enoate,

methyl 2-(4-(4-trifluoromethylphenoxy)phenoxy)propionate (DE-A 24 33 067);

E2) “Mononuclear” heteroaryloxyphenoxyalkanecarboxylic acid derivatives, for example

ethyl 2-(4-(3,5-dichloropyridyl-2-oxy)phenoxy)propionate (EP-A 0 002 925),

propargyl 2-(4-(3,5-dichloropyridyl-2-oxy)phenoxy)propionate (EP-A 0 003 114),

methyl 2-(4-(3-chloro-5-trifluoromethyl-2-pyridyloxy)phenoxy)propionate (EP-A 0 003 890),

ethyl 2-(4-(3-chloro-5-trifluoromethyl-2-pyridyloxy)phenoxy)propionate (EP-A 0 003 890),

propargyl 2-(4-(5-chloro-3-fluoro-2-pyridyloxy)phenoxy)propionate (EP-A 0 191 736),

butyl 2-(4-(5-trifluoromethyl-2-pyridyloxy)phenoxy)propionate (fluazifop-butyl);

E3) “Binuclear” heteroaryloxyphenoxyalkanecarboxylic acid derivatives, for example

methyl and ethyl 2-(4-(6-chloro-2-quinoxalyloxy)phenoxy)propionate (quizalofopmethyl and quizalofopethyl),

methyl 2-(4-(6-fluoro-2-quinoxalyloxy)phenoxy)propionate (see J. Pest. Sci. Vol. 10, 61 (1985)),

2-isopropylideneaminooxyethyl 2-(4-(6-chloro-2-quinoxalyloxy)phenoxy)propionate (propaquizafop),

ethyl 2-(4-(6-chlorobenzoxazol-2-yloxy)phenoxy)propionate (fenoxaprop-ethyl), its

D(+) isomer (fenoxaprop-P-ethyl) and ethyl 2-(4-(6-chlorobenzothiazol-2-yloxy)phenoxy)propionate (DE-A 26 40 730),

tetrahydro-2-furylmethyl 2-(4-(6-chloroquinoxalyloxy)phenoxy)propionate (EP-A 0 323 727);

F) Chloroacetanilides, for example

N-methoxymethyl-2,6-diethylchloroacetanilide (alachlor),

N-(3-methoxyprop-2-yl)-2-methyl-6-ethylchloroacetanilide (metolachlor),

2,6-dimethyl-N-(3-methyl-1,2,4-oxadiazol-5-ylmethyl)chloroacetanilide,

N-(2,6-dimethylphenyl)-N-(1-pyrazolylmethyl)chloroacetamide (metazachlor);

G) Thiocarbamates, for example

S-ethyl N,N-dipropylthiocarbamate (EPTC),

S-ethyl N,N-diisobutylthiocarbamate (butylate);

H) Cyclohexanedione oximes, for example

methyl 3-(1-allyloxyiminobutyl)-4-hydroxy-6,6-dimethyl-2-oxocyclohex-3-enecarboxylate (alloxydim),

2-(1-ethoxyiminobutyl)-5-(2-ethylthiopropyl)-3-hydroxycyclohex-2-en-1-one (sethoxydim),

2-(1-ethoxyiminobutyl)-5-(2-phenylthiopropyl)-3-hydroxycyclohex-2-en-1-one (cloproxydim),

2-(1-(3-chloroallyloxy)iminobutyl)-5-(2-ethylthiopropyl)-3-hydroxycyclohex-2-en-1-one,

2-(1-(3-chloroallyloxy)iminopropyl)-5-(2-ethylthiopropyl)-3-hydroxycyclohex-2-en-1-one (clethodim),

2-(1-ethoxyiminobutyl)-3-hydroxy-5-(thian-3-yl)cyclohex-2-enone (cycloxydim),

2-(1-ethoxyiminopropyl)-5-(2,4,6-trimethylphenyl)-3-hydroxycyclohex-2-en-1-one (tralkoxydim);

I) Benzoylcyclohexanediones, for example

2-(2-chloro-4-methylsulfonylbenzoyl)cyclohexane-1,3-dione (SC-0051, EP-A 0 137 963),

2-(2-nitrobenzoyl)-4,4-dimethylcyclohexane-1,3-dione (EP-A 0 274 634),

2-(2-nitro-4-methylsulfonylbenzoyl)-4,4-dimethylcyclohexane-1,3-dione (WO 91/13548, mesotrione);

J) S-(N-Aryl-N-alkylcarbamoylmethyl) dithiophosphonates such as S-[N-(4-chlorophenyl)-N-isopropylcarbamoylmethyl]O,O-dimethyl dithiophosphate (anilophos).

K) Alkylazines, for example as described in WO-A 97/08156, WO-A-97/31904, DE-A-19826670, WO-A-98/15536, WO-A-98/15537, WO-A-98/15538, WO-A-98/15539 and also DE-A-19828519, WO-A-98/34925, WO-A-98/42684, WO-A-99/18100, WO-A-99/19309, WO-A-99/37627 and WO-A-99/65882, preferably those of the formula (E)

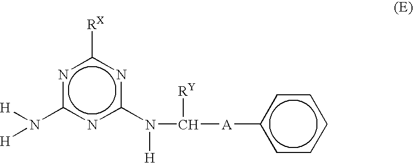

in which

R^{X} is (C₁-C₄)alkyl or (C₁-C₄)haloalkyl;

R^{Y} is (C₁-C₄)alkyl, (C₃-C₆)cycloalkyl or (C₃-C₆)cycloalkyl-(C₁-C₄)alkyl and

A is —CH₂—, —CH₂—CH₂—, —CH₂—CH₂—CH₂—, —O—, —CH₂—CH₂—O—, —CH₂—CH₂—CH₂—O—, especially preferably those of the formulae E I-E VII

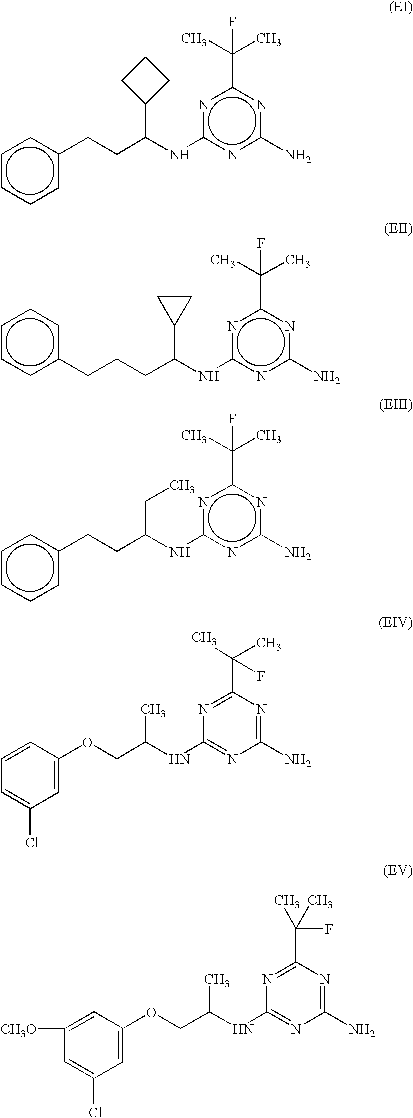

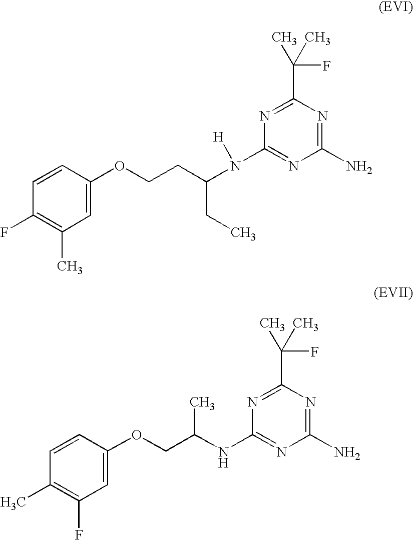

L) Phosphorus-containing herbicides, for example of the glufosinate type, such as glufosinate in the narrow sense, i.e. D,L-2-amino-4-[hydroxy(methyl)phosphinyl]butanoic acid, glufosinate monoammonium salt, L-glufosinate, L- or (2S)-2-amino-4-[hydroxy(methyl)phosphinyl]butanoic acid, L-glufosinatemonoammonium salt or bialaphos (or bilanafos), i.e. L-2-amino-4-[hydroxy(methyl)phosphinyl]butanoyl-L-alanyl-L-alanine, in particular its sodium salt, or of the glyphosate type, such as glyphosate, i.e. N-(phosphonomethyl)glycine, glyphosate monoisopropylammonium salt, glyphosate sodium salt, or sulfosate, i.e. N-(phosphonomethyl)glycine trimesium salt=N-(phosphonomethyl)glycine trimethylsulfoxonium salt.

The herbicides of groups B to L are known, for example, from each of the specifications stated above and from “The Pesticide Manual”, 12^{th} Edition, 2000, The British Crop Protection Council, “Agricultural Chemicals Book II—Herbicides—”, by W. T. Thompson, Thompson Publications, Fresno Calif., USA 1990 and “Farm Chemicals Handbook '90”, Meister Publishing Company, Willoughby Ohio, USA, 1990.

In a preferred embodiment, the agrochemical compositions according to the invention comprise one or more active substances selected from the group consisting of diflufenican, fenoxaprop-P-ethyl, metamitron, ethofumesate, phenmedipham, desmedipham or the abovementioned group L) of the phosphorus-containing herbicides, for example glufosinate-ammonium or glyphosate.

In addition to the adjuvants according to the invention and one or more agrochemical active substances, the agrochemical compositions according to the invention may also comprise further components, for example formulation auxiliaries such as anti-drift agents, substances for influencing moisture (humectants), fertilizers such as ammonium sulfate, urea or compound fertilizers, for example phosphorus-, potassium- and nitrogen-based compound fertilizers, such as P,K,N fertilizers, or commercially available surfactants other than those of the formula (I), such as nonionic, cationic, anionic, betainic or polymeric surfactants, stabilizers such as pH stabilizers, biocides, UV stabilizers, antifoams, synthetic or natural polymers, solvents, for example polar solvents such as waters or alcohols, or unpolar solvents such as saturated or unsaturated aliphatic solvents, which may be branched or unbranched, or aromatic solvents, such as Solvesso® 100, Solvesso® 150 or Solvesso® 200 or xylene. These agrochemical compositions and their use are likewise novel and subject-matter of the present invention.

The agrochemical compositions according to the invention have an outstanding agrochemical activity against harmful organisms such as harmful plants. The improved control of the harmful plants by the agrochemical compositions according to the invention makes it possible to reduce the application rate and/or to increase the safety margin. Both make sense both from the economical and the ecological angle.

In a preferred embodiment, agrochemical compositions according to the invention are characterized by a synergistically active content of a combination of the surfactants a) with fillers b) and agrochemical active substances c). In this context, it must be emphasized in particular that, as a rule, the agrochemical compositions of the invention have an inherent synergistic action, even in combinations with application rates or weight ratios of a):b):c) where synergism cannot be detected readily in each individual case, for example because the individual compounds are usually employed in very different application rates in the combination or else because even the individual compounds alone effect very good control of the harmful plants. The agrochemical compositions according to the invention can be prepared by customary processes, for example mixing by grinding, dissolving or dispersing the individual components, preferably at room temperature. This may be followed by, for example, an extrusion step such as a melt extrusion step. If other auxiliaries are present, they are preferably likewise incorporated at room temperature. In general, the sequence in which the individual components are added is of no decisive importance.

The components a), b) and c) of the agrochemical compositions according to the invention may be present together in a readymix which can then be applied in the customary fashion, for example in the form of a spray mixture, or they can be formulated separately and applied for example together by the tank mix method or separately, for example in succession. When the components are formulated separately, components a), b) and c) can be formulated for example in each case individually, or else components a) and b), a) and c) or b) and c) can be formulated jointly and the third component in each case is formulated separately.

By choosing suitable auxiliaries and/or preparation processes, adjuvants according to the invention are obtained which disintegrate well in the spray tank and which are also economical in use.

The adjuvants according to the present invention can be formulated in various ways, for example as granules, powders or dusts. Suitable possibilities of formulations are, for example, wettable powders (WP), water-soluble powders (SP), water-dispersible granules (WG), water-soluble granules (SG) and melt granules. Granules such as melt granules are preferred. The agrochemical compositions according to the invention and the agrochemical active substances c) can be formulated in various ways, depending on the prevailing biological and/or chemico-physical parameters. Suitable possibilities of formulations are, for example, wettable powders (WP), water-soluble powders (SP), water-soluble concentrates (SL), emulsifiable concentrates (EC), microemulsions (ME), emulsions (EW) such as oil-in-water and water-in-oil emulsions, sprayable solutions, suspension concentrates (SC), suspoemulsion concentrates (SE), oil- or water-based dispersions, oil-miscible solutions, capsule suspensions (CS), dusts (DP), seed-dressing products, granules for broadcasting and soil application, granules (GR) in the form of microgranules, spray granules, coated granules and adsorption granules, water-dispersible granules (WG), water-soluble granules (SG), ULV formulations, microcapsules and waxes. The preparation processes and formulation types are known in principle and are described, for example, in: Winnacker-Küchler, “Chemische Technologie” [Chemical Technology], Volume 7, C. Hauser Verlag Munich, 4^{th} Ed. 1986, Wade van Valkenburg, “Pesticide Formulations”, Marcel Dekker, N.Y., 1973; K. Martens, “Spray Drying” Handbook, 3rd Ed. 1979, G. Goodwin Ltd. London; H. Mollet, A. Grubenmann, “Formulierungstechnik” [Formulation Technology], Wiley-VCH, Weinheim 2000.

The formulation auxiliaries, such as inert materials, surfactants, solvents and further additives, are also known and are described, for example, in Watkins, “Handbook of Insecticide Dust Diluents and Carriers”, 2nd Ed., Darland Books, Caldwell N.J., H. v. Olphen, “Introduction to Clay Colloid Chemistry”; 2nd Ed., J. Wiley & Sons, N.Y.; C. Marsden, “Solvents Guide”; 2nd Ed., Interscience, N.Y. 1950; McCutcheon's “Detergents and Emulsifiers Annual”, MC Publ. Corp., Ridgewood N.J.; Sisley and Wood, “Encyclopedia of Surface Active Agents”, Chem. Publ. Co. Inc., N.Y. 1964; Schönfeldt, “Grenzflächenaktive Äthylenoxidaddukte” [Surface-active ethylene oxide adducts], Wiss. Verlagsgesellschaft, Stuttgart 1976; Winnacker-Küchler, “Chemische Technologie”, Volume 7, C. Hauser Verlag Munich, 4^{th} Ed. 1986.

Based on these formulations, it is also possible to prepare combinations with other agrochemical active substances such as herbicides, fungicides, insecticides, safeners, fertilizers and/or growth regulators, for example in the form of a readymix or a tank mix.

Wettable (sprayable) powders are products which are uniformly dispersible in water and which, besides the components a), b) and c), optionally comprise diluents or inert materials and further ionic and/or nonionic surfactants (wetters, dispersants), for example polyoxyethylated alkylphenols, polyoxyethylated fatty alcohols or fatty amines, alkanesulfonates or alkylbenzenesulfonates, sodium lignosulfonate, sodium 2,2′-dinaphthylmethane-6,6′-disulfonate, sodium dibutylnaphthalenesulfonate or else sodium oleoylmethyltauride. To prepare the wettable powders, the components a) and b) are ground finely, for example in customary apparatuses such as hammer mills, blower mills and air-jet mills, and mixed with the formulation auxiliaries, either simultaneously or subsequently.

Dusts are obtained by grinding the components a) and c) with finely divided solid substances such as the fillers b).

Granules may be, for example, granules for broadcasting and soil application, granules (GR) in the form of microgranules, spray granules, coated granules and adsorption granules, or water-dispersible granules (WG). They may be prepared for example by spraying the active substance onto adsorptive, granulated inert material or by applying active substance concentrates to the surface of carriers such as sand, kaolinites or granulated inert material, using adhesives, for example polyvinyl alcohol, sodium polyacrylate or else mineral oils. Suitable active substances may also be granulated in the fashion which is suitable for the production of fertilizer granules, if desired as a mixture of fertilizers. Thus, the agrochemical active substances may be ground for example with the surfactants of the formula (I) and the fillers b) and, if appropriate, further auxiliaries, the mixture may be subjected to melt extrusion and the extrudate may subsequently be granulated to give the desired particle size. Water-dispersible granules are generally prepared by the customary methods such as spray drying, fluidized-bed granulation, disk granulation, mixing with high-speed mixers and extrusion without solid inert material. As regards the preparation of disk granules, fluidized bed granules, extruder granules and spray granules, see, for example, methods in “Spray-Drying Handbook” 3rd ed. 1979, G. Goodwin Ltd., London; J. E. Browning, “Agglomeration”, Chemical and Engineering 1967, pages 147 et seq. “Perry's Chemical Engineer's Handbook”, 5th Ed., McGraw-Hill, New York 1973, p. 8-57.

Emulsifiable concentrates are prepared by dissolving the surfactant a) and/or agrochemical active substance c) in an organic solvent, for example butanol, cyclohexanone, dimethylformamide, xylene or else higher-boiling aromatics or hydrocarbons or mixtures of the organic solvents with addition of one or more ionic or nonionic surfactants (emulsifiers). Examples of emulsifiers which may be used are: calcium salts of alkylarylsulfonic acid, such as calcium dodecylbenzenesulfonate, or nonionic emulsifiers such as fatty acid polyglycol esters, alkylaryl polyglycol ethers, fatty alcohol polyglycol ethers, propylene oxide/ethylene oxide condensates, alkyl polyethers, sorbitan esters such as, for example, sorbitan fatty acid esters, or polyoxyethylene sorbitan esters such as, for example, polyoxyethylene sorbitan fatty acid esters.

Suspension concentrates can be water- or oil-based. They can be prepared, for example by wet grinding by means of commercially available bead mills and, if appropriate, addition of further surfactants as have already been mentioned for example above in the case of the other formulation types.

Emulsions, for example oil-in-water emulsions (EW), can be prepared for example by means of stirrers, colloid mills and/or static mixers using aqueous organic solvents and, if appropriate, further surfactants as have already been mentioned for example above in the case of the other formulation types.

For further details on the formulation of crop protection products, see, for example, G. C. Klingman, “Weed Control as a Science”, John Wiley and Sons, Inc., New York, 1961, pages 81-96 and J. D. Freyer, S. A. Evans, “Weed Control Handbook”, 5th Ed., Blackwell Scientific Publications, Oxford, 1968, pages 101-103.

In addition, the abovementioned active substance formulations may comprise, if appropriate, additives such as adhesives, wetters, dispersants, emulsifiers, penetrants, preservatives, antifreeze agents, solvents, fillers, carriers, colorants, antifoams, evaporation inhibitors, pH regulators or viscosity regulators which are customary in each case.

For use, the formulations, which are present in commercially available form, are, if appropriate, diluted in the customary manner, for example using water in the case of wettable powders and water-dispersible granules. Preparations in the form of dusts and granules for soil or broadcasting application are conventionally not diluted any further with other inert substances prior to use.

The agrochemical compositions according to the invention can be employed for example by application to the harmful organisms or the locations at which they occur, for example by spraying. The agrochemical active substances c) to be used in accordance with the invention are generally applied together with the surfactants a) and fillers b), or in succession, preferably in the form of a spray mixture comprising the surfactants a), fillers b), and the agrochemical active substances c) in effective amounts and, if appropriate, further customary auxiliaries. Preparation of the spray mixture is preferably based on water and/or an oil, for example a vegetable oil, or a high-boiling hydrocarbon such as kerosene or paraffin. The agrochemical compositions according to the invention can be realized for example as a tank mix or via a coformulation.

As a rule, the agrochemical formulations comprise from 0.1 to 99 percent by weight, in particular 2 to 95% by weight, of agrochemical active substances, the following concentrations being generally customary, depending on the formulation type: in wettable powders, the active substance concentration generally amounts to approximately 10 to 90% by weight, the remainder to 100% by weight being composed of customary formulation constituents. In the case of emulsifiable concentrates, the active substance concentration can amount to approximately 1 to 90% by weight, preferably 5 to 80% by weight. Formulations in the form of dusts comprise 1 to 30% by weight of active substance, preferably in most cases 5 to 20% by weight of active substance, while sprayable solutions comprise approximately 0.05 to 80, preferably 2 to 50,% by weight of active substance. In the case of water-dispersbile granules, the active substance content depends partly on whether the active compound is present in liquid or solid form and on the granulation aids, fillers and the like which are being used. In the case of the water-dispersible granules, the active substance content is, for example, between 1 and 95% by weight, preferably between 10 and 80% by weight. The percentages stated above may also be lower or higher in individual cases.

The spray mixture is preferably made in such a way that it is based on water. The weight ratio of surfactant (I): agrochemical active substance may vary within wide ranges. As a rule, it is generally in the range of from 5000:1 to 1:1000, preferably 2000:1 to 1:50, especially preferably 1000:1 to 1:2. The application rates of surfactant of the formula (I) are generally between 10 and 5000 g/ha, preferably between 50 and 2000 g/ha. However, the limits stated above may also be lower or higher in individual cases.

The concentration of surfactants of the formula (I) in the spray mixture is, as a rule, between 0.001 and 4% by weight, preferably 0.01 and 2% by weight. The concentration of fillers generally amounts to between 0.001 and 4% by weight, preferably 0.01 and 2% by weight.

The abovementioned preparation process for the adjuvants according to the invention also permit in principle the incorporation of agrochemical active substances, so that agrochemical compositions, for example in the form of granules, powders or dusts, which, in addition to the adjuvants according to the invention, also comprise one or more agrochemical active substances, can be obtained.

The agrochemical active substances which can be formulated together with the surfactants of the formula (I) are preferably compounds with a melting point of greater than 80° C. In individual cases, however, the melting point of the agrochemical active substances may also be less than 80° C.

The weight ratio between the surfactants of the formula (I) and the agrochemical active substances generally varies between 5000:1-1:1000, but may in individual cases also be lower or higher than the abovementioned limits. In a very especially preferred embodiment, the weight ratio is in the range of from 10:1 to 1:2, in particular 5:1 to 1:1.5. These above-described solid products such as granules, powders or dusts comprising adjuvants according to the invention and agrochemical active substances are likewise subject matter of the present invention.

The agrochemical compositions according to the invention are preferably herbicidal compositions which have an outstanding herbicidal activity against a broad spectrum of economically important monocotyledonous and dicotyledonous harmful plants. The active ingredients also act efficiently on perennial weeds which produce shoots from rhizomes, rootstocks or other perennial organs and which are difficult to control. In this context, it does not matter whether the substances are applied before sowing, pre-emergence or post-emergence. Specific examples may be mentioned of some representatives of the monocotyledonous and dicotyledonous weed flora which can be controlled by the compounds according to the invention, without the enumeration being a restriction to certain species.

Examples of weed species on which the herbicidal compositions act efficiently are, from amongst the monocotyledonous weed species, for example Apera spica venti, Avena spp., Alopecurus spp., Brachiaria spp., Digitaria spp., Lolium spp., Echinochloa spp., Panicum spp., Phalaris spp., Poa spp., Setaria spp. and Bromus spp. such as Bromus catharticus, Bromus secalinus, Bromus erectus, Bromus tectorum and Bromus japonicus, and Cyperus species from the annual group, and, among the perennial species, Agropyron, Cynodon, Imperata and Sorghum and also perennial Cyperus species.

In the case of the dicotyledonous weed species, the spectrum of action extends to genera such as, for example, Abutilon spp., Amaranthus spp., Chenopodium spp., Chrysanthemum spp., Galium spp. such as Galium aparine, Ipomoea spp., Kochia spp., Lamium spp., Matricaria spp., Pharbitis spp., Polygonum spp., Sida spp., Sinapis spp., Solanum spp., Stellaria spp., Veronica spp. and Viola spp., Xanthium spp., among the annuals, and Convolvulus, Cirsium, Rumex and Artemisia in the case of the perennial weeds.

The compositions according to the invention also act outstandingly efficiently on harmful plants which are found under the specific cultures in rice, such as, for example, Echinochloa, Sagittaria, Alisma, Eleocharis, Scirpus and Cyperus.

If the herbicidal compositions according to the invention are applied to the soil surface before germination, the weed seedlings are either prevented completely from emerging or else the weeds grow until they have reached the cotyledon stage, but then their growth stops, and, eventually, after three to four weeks have elapsed, they die completely.

If the herbicidal compositions according to the invention are applied post-emergence to the green parts of the plants, growth likewise stops drastically a very short time after the treatment, and the weed plants remain at the growth stage at the point in time of application, or they die completely after a certain time, so that in this manner competition by the weeds, which is harmful to the crop plants, is eliminated very early and in a sustained manner.

The herbicidal compositions according to the invention are distinguished by a rapidly commencing and long-lasting herbicidal action. As a rule, the rainfastness of the active substances in the combinations according to the invention is advantageous. A particular advantage is that the dosages used in the herbicidal compositions and the effective dosages of herbicidal compounds can be adjusted to such low a level that their soil action is optimally low. This does not only allow them to be employed in sensitive crops in the first place, but groundwater contaminations are virtually avoided. The active substance combination according to the invention allows the application rate of the active substances to be reduced considerably.

When adjuvants according to the invention and agrochemical active substances, in particular herbicides, are used jointly, superadditive (=synergistic) effects are observed in the preferred embodiment. This means that the effect in the combinations exceeds the expected total of the effects of the individual components employed. The synergistic effects allow the application rate to be reduced, a broader spectrum of broad-leaved weeds and grass weeds to be controlled, the herbicidal effect to commence more rapidly, the duration of action to be longer, the harmful plants to be controlled better while using only one, or few, applications, and the application window to be extended. In some cases, use of the compositions also reduces the amount of harmful constituents, such as nitrogen or oleic acid, and their entry into the ground.

The abovementioned properties and advantages are necessary for weed control practice to keep agricultural crops free from undesired competing plants, and thus to ensure and/or increase yield levels from the qualitative and quantitative angle. These novel combinations markedly exceed the technical state of the art with a view to the properties described.

While the compositions according to the invention have an outstanding herbicidal activity against monocotyledonous and dicotyledonous weeds, crop plants of economical important crops, for example dicotyledonous crops such as soya, cotton, oilseed rape, sugar beet, or graminacous crops such as wheat, barley, rye, oats, sorghum and millet, rice or maize, are damaged only to a minor extent, if at all. This is why the present compounds are highly suitable for the selective control of undesired plant growth in plantations of agricultural crops or of ornamentals.

In addition, some of the compositions according to the invention have outstanding growth-regulatory properties in crop plants. They engage in the plants' metabolism in a regulatory manner and can thus be employed for provoking direct effects on plant constituents and to facilitate harvesting such as, for example, by triggering desiccation and stunted growth. Moreover, they are also suitable for the general control and inhibition of undesired vegetative growth without simultaneously destroying the plants. Inhibition of vegetative growth is very important in a large number of monocotyledonous and dicotyledonous crops since yield losses caused by lodging can thus be reduced, or prevented completely.

Owing to their herbicidal and plant-growth-regulatory properties, the compositions according to the invention can be employed for controlling harmful plants in crop plants which have been genetically modified or obtained by mutation selection. These crop plants are generally distinguished by specific advantageous characteristics, such as resistances to herbicidal compositions or resistances to plant diseases or the causative organisms of plant diseases such as specific insects or microorganisms such as fungi, bacteria or viruses. Other specific characteristics relate, for example, to the harvested material with regard to quantity, quality, storability, composition and specific constituents. Thus, for example, transgenic plants are known whose starch content is increased, or whose starch quality is altered, or those where the harvested material has a different fatty acid composition.

The use of the compositions according to the invention in economically important transgenic crops of useful plants and ornamentals, for example of graminacous crops such as wheat, barley, rye, oats, sorghum and millet, rice and maize, or else crops of sugar beet, cotton, soya, oilseed rape, potatoes, tomatoes, peas and other vegetables, is preferred. Preferably, the compositions according to the invention can be employed as herbicides in crops of useful plants which resist the phytotoxic effects of the herbicides, or have been made to resist these effects by recombinant techniques.

When using the herbicidal compositions according to the invention in transgenic crops, effects are frequently observed in addition to the effects against harmful plants to be observed in other crops, which are specific for the application in the transgenic crop in question, for example a modified or specifically widened weed spectrum which can be controlled, modified application rates which may be employed for application, preferably good combining ability with the herbicides to which the transgenic crop is resistant, and an effect on growth and yield level of the transgenic crop plants.

The present invention therefore furthermore also relates to a method for controlling undesired vegetation, preferably in crops of plants such as cereals (for example wheat, barley, rye, oats, rice, maize, sorghum and millet), sugar beet, sugar cane, oilseed rape, cotton and soya, especially preferred in monocotyledonous plants such as cereals, for example wheat, barley, rye, oats, and their hybrids such as triticale, rice, maize, sorghum and millet, where one or more herbicidal compositions according to the invention are applied to the harmful plants, plant parts, seeds of the plants or the area on which the plants grow, for example the area under cultivation.

The plant crops may also be genetically modified or have been obtained by mutation selection; they preferably tolerate acetolactate synthase (ALS) inhibitors.

The invention therefore also relates to the use of the herbicidal compositions according to the invention for controlling harmful plants, preferably in plant crops.

The herbicidal compositions according to the invention can also be employed nonselectively for controlling undesired vegetation, for example in plantation crops, on verges, squares, industrial terrain or rail tracks.

The agrochemical compositions according to the invention, in particular herbicidal compositions, can exist not only as mixed formulations, if appropriate together with other agrochemical active substances, and auxiliaries such as additives and/or formulation aids, which are then diluted with water and applied as usual, but also as what are known as tank mixes by jointly diluting, with water, the components which have been formulated separately or partially separately.

Owing to the relatively low application rate of the herbicidal compositions according to the invention, they are generally very well tolerated. In particular, a reduction in the absolute application rate can be achieved by the combinations according to the invention, compared with the individual use of a herbicidal active substance.

The invention therefore also relates to a method of controlling harmful plants, preferably for the selective control of harmful plants in plant crops, which comprises applying, for example pre-emergence, post-emergence or pre- and post-emergence, preferably pre-emergence, a herbicidally active amount of the abovementioned herbicides c) in combination with at least one of the surfactants a) and at least one filler b) to the plants, plant parts, seeds of the plants or the area on which the plants grow, for example the area under cultivation, either jointly or in succession.

In a preferred method variant, the herbicides c) are applied at application rates of from 0.1 to 2000 g of active substance/ha, especially preferably 0.5 to 1000 g of active substance/ha. It is furthermore especially preferred to apply the active substances in the form of a coformulation or in the form of tank mixes, where the individual components, for example in the form of formulations, are jointly mixed in the tank with water and the resulting spray mixture is applied.

Since the crop plant compatibility of the combinations according to the invention is extremely good, combined with a very high degree of control of the harmful plants, the combinations according to the invention can be considered as selective. In a preferred embodiment, herbicidal compositions with the active substance combinations according to the invention are therefore employed for selectively controlling undesired plants.

If, if desired, the compatibility and/or selectivity of the herbicidal compositions according to the invention is to be increased even further, it may be advantageous to apply them together with safeners or antidotes, either jointly in a mixture or staggered in time.

Compounds which are suitable as safeners or antidotes for the herbicidal compositions according to the invention are known, for example, from EP-A-333 131 (ZA-89/1960), EP-A-269 806 (U.S. Pat. No. 4,891,057), EP-A-346 620 (AU-A-89/34951) and the international patent applications PCT/EP 90/01966 (WO-91108202) and PCT/EP 90102020 (WO-911078474) and literature cited therein or can be prepared by the processes described therein. Further suitable safeners are known from EP-A-94 349 (U.S. Pat. No. 4,902,304), EP-A-191 736 (U.S. Pat. No. 4,881,966) and EP-A-0 492 366 and the literature cited therein.

In a preferred embodiment, the herbicidal compositions of the present invention therefore contain an additional content of one or more compounds which act as safeners or antidotes.

Especially preferred antidotes or safeners or groups of compounds which are suitable as safeners or antidotes for the above-described herbicidal compositions of the invention are, inter alia:

a) compounds of the dichlorophenylpyrazoline-3-carboxylic acid type, preferably compounds such as ethyl 1-(2,4-dichlorophenyl)-5-(ethoxycarbonyl)-5-methyl-2-pyrazoline-3-carboxylate (compound S1-1, mefenpyr-diethyl) and related compounds as are described in the international application WO 91/07874 (PCT/EP 90102020);

b) dichlorophenylpyrazolecarboxylic acid derivatives, preferably compounds such as ethyl 1-(2,4-dichlorophenyl)-5-methylpyrazole-3-carboxylate (compound S1-2), ethyl 1-(2,4-dichlorophenyl)-5-isopropylpyrazole-3-carboxylate (compound S1-3), ethyl 1-(2,4-dichlorophenyl)-5-(1,1-dimethylethyl)pyrazole-3-carboxylate (compound S1-4), ethyl 1-(2,4-dichlorophenyl)-5-phenylpyrazole-3-carboxylate (compound S1-5) and related compounds as are described in EP-A-0 333 131 and EP-A-0 269 806;

c) compounds of the triazolecarboxylic acid type, preferably compounds such as ethyl 1-(2,4-dichlorophenyl)-5-trichloromethyl-(1H)-1,2,4-triazole-3-carboxylate (compound S1-6, fenchlorazole) and related compounds (see EP-A-0 174 562 and EP-A-0 346 620);

d) compounds of the dichlorobenzyl-2-isoxazoline-3-carboxylic acid type, compounds of the 5-benzyl- or 5-phenyl-2-isoxazoline-3-carboxylic acid type, preferably compounds such as ethyl 5-(2,4-dichlorobenzyl)-2-isoxazoline-3-carboxylate (compound S1-7) or ethyl 5-phenyl-2-isoxazoline-3-carboxylate (compound S1-8), and related compounds as are described in international patent application WO 91/08202 (PCT/EP 90/01966);

e) compounds of the 8-quinolinoxyacetic acid type, preferably compounds such as 1-methylhex-1-yl (5-chloro-8-quinolinoxy)acetate (S2-1; cloquintocet-mexyl), 1,3-dimethylbut-1-yl (5-chloro-8-quinolinoxy)acetate (S2-2), 4-allyloxybutyl (5-chloro-8-quinolinoxy)acetate (S2-3), 1-allyloxyprop-2-yl (5-chloro-8-quinolinoxy)acetate (S2-4), ethyl (5-chloro-8-quinolinoxy)acetate (S2-5), methyl (5-chloro-8-quinolinoxy)acetate (S2-6), allyl (5-chloro-8-quinolinoxy)acetate (S2-7), 2-(2-propylideneiminoxy)-1-ethyl (5-chloro-8-quinolinoxy)acetate (S2-8), 2-oxoprop-1-yl (5-chloro-8-quinolinoxy)acetate (S2-9) and related compounds as are described in EP-A-0 086 750, EP-A-0 094 349 and EP-A-0 191 736 or EP-A-0 492 366;

f) compounds of the (5-chloro-8-quinolinoxy)malonic acid type, preferably compounds such as diethyl (5-chloro-8-quinolinoxy)malonate, diallyl (5-chloro-8-quinolinoxy)malonate, methylethyl (5-chloro-8-quinolinoxy)malonate and related compounds as have been described and proposed in German patent application EP-A-0 582 198;

g) active substances of the type of the phenoxyacetic acid derivatives or phenoxypropionic acid derivatives or of the aromatic carboxylic acids such as, for example, 2,4-dichlorophenoxyacetic acid (and esters) (2,4-D), 4-chloro-2-methylphenoxypropionic acid (mecoprop), MCPA or 3,6-dichloro-2-methoxybenzoic acid (and esters) (dicamba).

h) compounds of the 5,5-diphenyl-2-isoxaoline-3-carboxylic acid type, preferably ethyl 5,5-diphenyl-2-isoxazoline-3-carboxylate (S3-1, isoxadifen-ethyl).

i) compounds which are known as safeners, for example for rice, such as fenclorim (=4,6-dichloro-2-phenylpyrimidine, Pesticide Manual, 11^{th} Edition, 1997, pp. 511-512), dimepiperate (=S-1-methyl-1-phenylethyl piperidine-1-thiocarboxylate, Pesticide Manual, 11^{th} Edition, 1997, pp. 404-405), daimuron (=1-(1-methyl-1-phenylethyl)-3-p-tolylurea, Pesticide Manual, 11^{th} Edition, 1997, p. 330), cumyluron (=3-(2-chlorophenylmethyl)-1-(1-methyl-1-phenylethyl)urea, JP-A-60/087254), methoxyphenone (=3,3′-dimethyl-4-methoxybenzophenone, CSB (=1-bromo-4-(chloromethylsulfonyl)benzene, CAS-Reg. No. 54091-06-4).

In addition, at least some of the abovementioned compounds are described in EP-A-0 640 587, which is herewith referred to for disclosure purposes.

j) A further important group of compounds which are suitable as safeners and antidotes is known from WO 95/07897.

The safeners (antidotes) of the above groups a) to j) reduce or prevent phytotoxic effects which may be observed when the herbicidal compositions according to the invention are employed in crops of useful plants, without adversely affecting the efficacy of the herbicides against harmful plants. This makes it possible considerably to widen the spectrum of application of the herbicidal compositions according to the invention; in particular, the use of safeners makes possible the application of herbicidal compositions which could previously only be employed to a limited extent or with insufficient success, i.e. of combinations which, at low dosages with a poor spectrum of action, led to insufficient control of the harmful plants without safener.

Components a), b) and c) of the herbicidal compositions according to the invention and the abovementioned safeners can be applied jointly (for example as readymix or by the tank mix method) or in succession in any desired sequence. The weight ratio safener:herbicide (compound(s) of the formula (I) and/or their salts) can vary within wide ranges and is preferably in the range of from 1:100 to 100:1, in particular of from 1:100 to 50:1. The amounts of herbicide(s) and safener(s) which are optimal in each case usually depend on the type of the herbicidal composition and/or on the safener used, and also on the nature of the plant stand to be treated.

Depending on their properties, the safeners can be used for pretreating the seed of the crop plant (seed dressing) or introduced into the seed furrows prior to sowing or applied together with the herbicide mixture before or after emergence of the plants. Pre-emergence treatment includes both the treatment of the area under cultivation before sowing and the treatment of the areas under cultivation where seed has been sown, but growth is as yet not present. The joint application with the herbicide mixture is preferred. Tank mixes or readymixes can be employed for this purpose.

The application rates required, of the safeners, can vary within wide limits, depending on the indication and the herbicide used; they are, as a rule, in the range of from 0.001 to 1 kg, preferably 0.005 to 0.2 kg, of active substance per hectare.

The herbicidal compositions according to the invention can be applied in the customary fashion, for example with water as carrier in spray mixture quantities of approximately 5 to 4000 liters/ha. Application of the compositions by what is known as the low-volume and ultra-low-volume methods (ULV) is also possible, as is their application in the form of granules and microgranules.

A preferred use relates to application of herbicidal compositions which contain components a), b) and c) in a synergistically active amount. The invention also extends to mixtures of one or more surfactants a) with one or more fillers b), and one or more herbicides c). In addition, one, two or more agrochemical active substances other than the herbicides c) (for example insecticides, fungicides, safeners) may be present in the herbicidal compositions of the invention for complementing their properties, usually in minor amounts.

This gives rise to numerous possibilities of combining several agrochemical active substances with each other and using them jointly to control harmful plants without deviating from the scope of the invention.

Thus, in a preferred embodiment, for example various herbicidal active substances of the formula (III) and/or their salts can be combined with each other, for example

mesosulfuron-methyl+iodosulfuron-methyl,

mesosulfuron-methyl+iodosulfuron-methyl-sodium,

mesosulfuron-methyl+foramsulfuron,

mesosulfuron-methyl+foramsulfuron-sodium,

mesosulfuron-methyl-sodium+iodosulfuron-methyl,

mesosulfuron-methyl-sodium+iodosulfuron-methyl-sodium,

mesosulfuron-methyl-sodium+foramsulfuron,

mesosulfuron-methyl-sodium+foramsulfuron-sodium,

foramsulfuron+iodosulfuron-methyl,

foramsulfuron+iodosulfuron-methyl-sodium,

foramsulfuron-sodium+iodosulfuron-methyl,

foramsulfuron-sodium+iodosulfuron-methyl-sodium.

The herbicidal active substances c) and their mixtures, for example the abovementioned active substance mixtures of active substances of the formula (III) and/or their salts, can be combined with one or more safeners, in particular with the safeners mefenpyr-diethyl (S1-1), cloquintocet-mexyl (S2-1) and isoxadifen-ethyl (S3-1).

The adjuvants according to the invention have high physical stability and a high surfactant load; moreover, they are ecologically advantageous. Furthermore, they are suitable for the preparation of agrochemical compositions with high biological activity, a high active substance load and an outstanding tank mix quality.

The use examples which follow illustrate the invention and are not limiting.

### EXAMPLES

### Preparation of the Adjuvants

### Example 1

A mixture of 50 g of Sapogenat® T500, 20 g of Sipernat® 50S, 10 g of Atlox® Metasperse 150S, 5 g of Morwet® EFW, 10 g of ammonium sulfate, 5 g of effervescent powder (citric acid/sodium hydrogencarbonate) is mixed intensively at temperatures of 80-100° C. in a kneader (LUK 025TV from Werner und Pfleiderer) (mixing time: 40 minutes). The mixture is subsequently transferred to a bench-top granulation apparatus (Benchtop KAR-75) and made into extrusion granules. Dispersibility and suspendability of the resulting samples showed very high quality in accordance with CIPAC measurement methods, even after storage of the samples for 2 weeks at 54° C.

The adjuvants listed in Table 1 hereinbelow (Examples 2-5) were obtained analogously in the form of granules (data in % by weight):

TABLE 1

| Adjuvant | 2 | 3 | 4 | 5 |
|---|---|---|---|---|
| Sapogenat ® T500 | 52.91 | 52.91 | 50.76 | 41.69 |
| Sipernat ® 508 | 17.99 | 17.99 | 21.32 | 17.20 |
| Tamol ® PP | 5.29 | — | — | — |
| Genapol ® LRO (paste, 69%) | 7.94 | 7.94 | 7.61 | 8.60 |
| Kaolin 1777 | 15.87 | — | — | — |
| Atlox ® Metasperse 150S | — | 5.29 | 5.08 | — |
| Steamic ® OOS | — | 15.87 | — | — |
| Morwet ® EFW | — | — | — | 5.06 |
| Ammonium sulfate | — | — | 15.23 | 27.40 |
| Fluowet ® PP | — | — | — | 0.05 |

### Example 6

31 g of Sapogenat® T500 pieces were ground in an IKA laboratory mill to a particle size of approx. 200 μm, and mixed intimately in a kitchen blender with 21.05 g of glufosinate-ammonium, 16.75 g of Sipernat® 350 (highly-disperse silica), 3.04 g of Atlox® Metasperse 150S, 5.10 g of Genapol® LRO (paste, 69%), 0.06 g of Fluowet® PP, 20 g of ammonium sulfate and 3 g of Agrimer® XLF.

This blend was metered into a melt extrusion apparatus from Thermo-Prism (type twin-screw extruder Eurolab 16 mm) using a vibrating chute and extruded through a multihole nozzle with 0.7 mm holes. The operating temperature was 25° C. in the drawing-in zone, 65° C. in all of the mixing and kneading zone, and 60° C. immediately before the nozzle. The extrudate solidified within a few seconds upon leaving the extruder and was broken into pieces approximately 3 mm in length. The dispersibility of the extrudate was measured in accordance with CIPAC MT174 and amounted to 70% by weight. This method is described in: CIPAC Handbook, Collaborative International Pesticides Analytical Council Ltd 1995: Physico-chemical Methods for Technical and Formulated Pesticides, Vol. F, page 435.

### Example 7

Example 6 was repeated with the only difference that glufosinate was used instead of glyphosate-ammonium as active substance. The dispersibility of the extrudate was 76% by weight.

### Example 8

900 g of Sapogenat® T500 were melted in a steam-heated 5 l mixer from Lödige. 269.6 g of Sipernat® 50S were introduced and the mixture was homogenized until a flowable absorbate was obtained. The heater was then switched off, and 618.4 g of diflufenican (previously ground to a particle size of 5 μm) were admixed. Then, 130 g of Genapol® X-150, 80 g of Atlox Metasperse® 150S and 2 g of Fluowet® PP were introduced and the mixture was homogenized. This mixture was melt-extruded as described in Example 6. The dispersibility of the extrudate was 69% by weight.

### Example 9

180.95 g of Sapogenat® T500 pieces were ground in an IKA laboratory mill to a particle size of 200 μm, and mixed intimately in a kitchen blender with 187.5 g of metamitron and an absorbate of 78.1 g of Genapol® LRO (paste, 69%) on 78.1 g of Sipernat® 50S, 18.35 g of Atlox® Metasperse® 150S, 5 g of Disintex® 200 and 0.35 g of Fluowet® PP. This mixture was melt-extruded as described in Example 6. The dispersibility of the extrudate was 71% by weight.

Notes:

| | |
|---|---|
| Sapogenat ® T500 | 2,4,6-tri-sec-butylphenyl polyethoxylate |
| | (Clariant) |
| Sipernat ® 50S, 350 | highly-disperse precipitated silica |
| | (Degussa) |
| Tamol ® PP | phenolsulfonic acidlformaldehyde |
| | polycondensate, sodium salt (BASF) |
| Genapol ® LRO (paste, 69%) | lauryl ethoxyl ether sulfate (Clariant) |
| Genapol ® X-1 50 | isotridecyl alcohol ethoxylate (Clariant) |
| Kaolin ® 1777 | clay (Ziegler, Wunsiedel) |
| Atlox Metasperse ® 150S | styrene/acrylic acid copolymer |
| | (Uniquema) |
| Steamic ® ODS | aluminum hydrosilicate (Talc de Luzenac) |
| Morwet ® EFW | alkylnaphthalenesulfonic acid |
| | derivative (Akzo Nobel) |
| Fluowet ® PP | perfluoroalkylphosphonic acid |
| | (Clariant) |
| Agrimer ® XLF | crosslinked polyvinylpyrrolidone (ISP) |
| Disintex ® 200 | crosslinked polyvinylpyrrolidone (ISP) |

What is claimed is:

1. A solid adjuvant comprising
a) one or more surfactants of the formula (I),
Ar—O—(CHR¹—CHR²—O—)_{y}—R³ (I)
where
Ar is aryl which is substituted by at least two (C₁-C₃₀)alkyl radicals,
R¹ is H or (C₁-C₆)alkyl,
R² is H or (C₁-C₆)alkyl,
R³ is H, an unsubstituted or substituted (C₁-C₃₀) hydrocarbon radical, a sulfonate radical, a phosphonate radical or an acyl radical, and
y is an integer from 1 to 100, and
b) one or more fillers.

2. A solid adjuvant as claimed in claim 1 wherein, in formula (I), y is an integer from 15 to 100.

3. A liquid adjuvant as claimed in claim 1, wherein, in formula (I),
Ar is a naphthyl or phenyl residue having 3-7 (C₁-C₁₀)alkyl radicals attached to it,
R¹ is H or methyl,
R² is H or methyl,
R³ is H, (C₁-C₃₀)alkyl, (C₂-C₃₀)alkenyl, (C₂-C₃₀)alkynyl, a sulfate radical, a phosphate radical or an acyl radical, and
y is an integer from 1 to 20.

4. A liquid adjuvant as claimed in claim 1, wherein the filler b) is an inorganic carrier or a synthetic carrier.

5. A solid adjuvant as claimed in claim 1, additionally comprising one or more auxiliaries conventionally used in crop protection.

6. A process for the preparation of a solid adjuvant as claimed in claim 1, wherein the components are mixed with each other.

7. The use of a solid adjuvant as claimed in one or more of claim 1 for the preparation of an agrochemical composition.

8. An agrochemical composition comprising
α) one or more solid adjuvants as defined in claim 1, and
β) one or more agrochemical active substances.

9. An agrochemical composition as claimed in claim 8, wherein the agrochemical active substance is a herbicide.

10. An agrochemical composition as claimed in claim 9, wherein the agrochemical active substances are selected from the group consisting of diflufenican, fenoxaprop-P-ethyl, metamitron, ethofumesate, phenmedipham, desmedipham or phosphorous containing herbicides.

11. An agrochemical composition as claimed in claim 8, additionally comprising one or more further components selected from the group of the agrochemical active substances and the auxiliaries conventionally used in crop protection.

12. A method for controlling harmful organisms, wherein the components of the agrochemical composition as defined in claim 8 are applied together or separately to the harmful organisms or the locations at which they occur.

13. A method for controlling harmful plants, wherein the components of the herbicidal composition as defined in claim 9 are applied together or separately to the plants, plant parts, seeds of the plants or the area on which the plants grow.

14. A method as claimed in claim 13 for the selective control of harmful plants in plant crops.

15. The use of the agrochemical composition defined in claim 8 for controlling harmful organisms.

16. The use of the herbicidal composition defined in claim 9 for controlling harmful plants.

17. A process for the preparation of an agrochemical composition as defined in f claim 8, wherein the components are mixed with each other.
